(19) United States
(12) Patent Application Publication
(10) Pub. No.: US 2026/0207713 A1
(43) Pub. Date: Jul. 23, 2026

(54) Drug Combination Kit for Controlling Blood Sugar

(71) Applicant: Ningbo Y Biotechology lnc., Ningbo (CN)

(72) Inventor: Qingming YU, Lianyungang (CN)

(73) Assignee: Ningbo Y Biotechology lnc., Ningbo (CN)

(21) Appl. No.: 19/439,449

(22) Filed: Jan. 4, 2026

Related U.S. Application Data
(63) Continuation of application No. PCT/CN2024/103218, filed on Jul. 3, 2024.

(30) Foreign Application Priority Data
Jul. 4, 2023 (CN) 202310808623.0

(51) Int. Cl. A61K 38/28; A61K 9/00; A61K 9/08; A61K 9/48; A61K 31/22; A61P 3/10

(52) U.S. Cl. CPC A61K 38/28; A61K 9/0053; A61K 9/08; A61K 9/4891; A61K 31/22; A61P 3/10

(57) ABSTRACT

A drug combination kit for controlling blood sugar, comprising oral insulin enteric-coated capsules and ketone ester oral liquid or ketone ester capsules. The oral insulin enteric-coated capsules are taken before bedtime to achieve good control of fasting blood sugar at night and in the morning. The ketone ester oral liquid is taken before meals during the day, so that the human body enters a ketogenic state, not only lowering the blood sugar, but also avoiding postprandial blood sugar peaks. The combination of the two drugs can achieve better control of the blood sugar in the human body. Furthermore, ketone esters can further enhance insulin sensitivity and improve the cardiac function of diabetic patients. The kit has low cost and good sugar control effect, and is currently an ideal sugar control solution for diabetic patients.

### RELATED APPLICATIONS

The present patent document is a continuation of PCT Application Serial No. PCT/CN2024/103218, filed Jul. 3, 2024, designating the United States and published in English, which is hereby incorporated by reference.

The present patent document claims the benefit of priority to patent application No. 202310808623.0, filed Jul. 4, 2023, and entitled “Drug Combination Kit for Controlling Blood Sugar,” the entire contents of each of which are incorporated herein by reference.

### BACKGROUND

### 1. Technical Field

The invention belongs to the medical field, specifically relating to a drug combination kit for blood sugar control and diabetes treatment.

### 2. Background Information

Insulin is extensively employed for the regulation of blood sugar in patients with diabetes. Especially in recent years, recombinant human insulin and mutants thereof, along with various insulin analogs such as long-acting, ultra-long-acting, and ultra-short-acting insulin, have become better means for blood sugar management for diabetes patients. Currently, the majority of insulin on the market is in injectable form, which can lead to numerous side effects. Among the top ten drugs causing adverse events, insulin ranks first with an incidence rate of 8%. Insulin injections frequently result in the side effects, mainly including hypoglycemia, effects on heart rate, weight gain, subcutaneous thickening at the injection sites, insulin allergic reactions, an increased incidence of rectal and colon cancers with long-term use, and hypertension in type II diabetes patients.

Due to the inconvenient administration and side effects of injectable formulations, there has been a century-long anticipation for oral formulations. Numerous scientists and biotechnology firms have continuously endeavored to develop oral formulations, but most efforts have ended in failure. Recently, the oral formulation developed by Oramed, an Israeli company, was regarded as the most promising for commercialization. However, the company announced the failure of Phase III clinical trials in January 2023. In contrast, Tianhui Biotechnology in China using the similar technology reported success in its phase III clinical trials in April 2023. Following the submission of its drug listing application, Tianhui Biotechnology's oral insulin is poised to become the first commercially viable medication, offering hope to humanity.

The success of Tianhui Biotechnology's technical solution is primarily attributable to differences in its clinical protocol compared to Oramed's. Tianhui Biotechnology's clinical protocol involved selecting patients who had not responded well to 1-2 types of oral hypoglycemic drugs. The eligible patients had a BMI index ranging from 18.5 to 30 and took 16 mg pills before meals daily, with a dosage of 1-3 pills per dose

Oramed's clinical protocol stipulated administering one 8 mg pill at bedtime each day. The selected patients demonstrated poor response to 2-3 types of chemical hypoglycemic drugs, with a BMI index ranging from 25 to 40. It is believed that the domestic clinical protocol can achieve success, given the selection of patients with relatively mild conditions, where lower body weight under identical conditions signifies milder disease states. Given that type II diabetes is predominantly due to obesity, the therapeutic efficacy of Tianhui Biotechnology's oral formulation on severely affected patients warrants further clinical validation. Issues such as the high dosage and cost of insulin mean that only individuals with higher incomes may be able to afford it upon commercialization. Although IGF receptors in intestinal cells have a very weak affinity to insulin, concerns about potential adverse effects due to long-term high-dose oral administration of insulin, particularly in the presence of these receptors, also warrant further clinical validation.

Under normal physiological conditions, insulin is secreted by the pancreas, enters the portal vein of the liver, passes through the hepatic tissue and eventually circulates through the bloodstream to the entire body. In the absence of food intake, basal insulin is secreted by the pancreas to stabilize blood sugar. Postprandial blood sugar spikes occur, with subsequent insulin secretion spikes from the pancreas aiming to mitigate these postprandial blood sugar spikes. The hepatic metabolism leads to an insulin gradient from secretion to systemic circulation, resulting in notably low peripheral insulin concentrations. Compared to injectable formulations, drugs enter the body and form high concentrations in the periphery before acting on the liver, leading to clinical side effects. Oral insulin enteric-coated capsules allow insulin to be absorbed through the intestines, enter the hepatic portal vein, and finally distribute throughout the body, somewhat mimicking natural insulin secretion in vivo, thus avoiding the side effects of insulin injections. Oramed's clinical protocol utilizes regular recombinant insulin, which is relatively low-cost and administered at bedtime. It is scientifically designed to be taken in a fasted state, maintaining good consistency in drug absorption. Clinical results indicate effective control of nighttime and morning fasting blood sugar levels but ineffective control of postprandial blood sugar spikes, leading to clinical failure. The clinical protocol of Tianhui Biotechnology in China demonstrates good consistency in drug absorption when administered on an empty stomach in the morning. However, due to daily variations in the types and amounts of food consumed before lunch and dinner, controlling absorption consistency is challenging. Nevertheless, effective postprandial blood sugar control is achieved using high doses, enabling success in Phase III clinical trials.

The traditional ketogenic diet is a low-carbohydrate diet, typically consisting of no more than 5% carbohydrates, 20-30% protein, and 70-80% fat per meal. It was initially used for the treatment of intractable epilepsy and also for advanced diabetes before insulin availability, potentially extending lifespan by 2-3 years. However, a low-carbohydrate diet can cause many side effects and generally needs to be carried out under the guidance of a doctor or dietitian for a short period. For most individuals, it is a struggle to sustain commitment to it for a longer duration. The recently developed exogenous ketones are favored as they do not require changes in dietary structure and can be consumed to induce ketosis. The early exogenous ketone was sodium 3-hydroxybutyrate, but it exhibited poor ketogenic effects, short duration, and excessive consumption may lead to electrolyte imbalance. MCT oil is another type of exogenous ketone that can be consumed over a long term, but its effectiveness is suboptimal, with 50% of users experiencing diarrhea as a side effect.

Ketoester is a new exogenous ketone, launched as a dietary supplement in the United States in 2016 under the trade name KE4; another American brand, HVMN, was launched in 2017; deltaG ketones were launched in Europe in 2020. This ketoester is an oral solution with a potent ketogenic effect, reaching approximately 3 mM, and has a long duration of 4-6 hours. It is currently the most potent exogenous ketone globally, with multiple effects such as anti-aging, anti-inflammation, anti-cancer, prevention of neurodegeneration, reducing blood sugar, enhancing insulin sensitivity, and most importantly, preventing postprandial blood sugar spikes. The Buck Institute in the United States is conducting anti-aging research. In addition, the United States is also conducting clinical trials for type I and II diabetes and cognitive improvement trials for individuals over 50 years old. Recently, the University of Oxford reported that patients with type II diabetes, who controlled blood sugar with chemical hypoglycemic drugs instead of injection of insulin, experienced a 13.4% reduction in fructosamine, representing the past month's blood sugar level, and an 8.3% reduction in glycated hemoglobin over 28 consecutive days. This suggests that ketoesters significantly aid in blood sugar improvement for diabetes patients, through a mechanism different from insulin.

### BRIEF SUMMARY

Based on the progress of oral insulin development and theoretical analysis of the advantages and disadvantages of both marketed and failed oral insulin formulations, the inventor proposes a superior drug combination, assembled into a reagent kit. The kit comprises oral insulin capsules and ketoester oral solution or ketoester soft capsules, and is anticipated to optimize blood sugar control in diabetes patients.

On one hand, the present invention provides a drug combination kit for diabetes patients aimed at controlling blood sugar levels. This kit includes oral insulin capsules and ketoester oral solution or ketoester soft capsules.

According to the present invention, the dosage of oral insulin capsules ranges from 2-20 mg, preferably 4-16 mg, and more preferably 6-10 mg. The dosage of ketoester oral solution ranges from 2-50 g, preferably 5-25 g, and more preferably 10-15 g. Alternatively, the dosage of ketoester soft capsules ranges from 0.5-5 g, preferably 3-4 g. The dosage in the present invention refers to the quantity of the active ingredient, for example, the dosage of oral insulin capsules is the quantity of insulin in the capsules. Similarly, the dosage of ketoester oral solution or ketoester soft capsules refers to the quantity of ketoesters present in the oral solution or soft capsules. Unless otherwise specified, the term “dosage” in the present invention refers to “unit dosage”. Generally, ketoesters are formulated into solutions of about 50% (w/v) or lower concentrations, such as 40%, 30%, 20%, 10%, etc. The primary component of gastric soluble soft capsules is gelatin, generally manufactured using plasticizers such as sorbitol or mannitol, and preservatives like methylparaben or ethylparaben, resulting in a soft and smooth texture. The palatability of the ketoester solution is not ideal. To improve patient compliance, the invention employs gastric soluble soft capsules to encapsulate ketoesters, enhancing the taste. Each soft capsule ideally has a dosage (i.e. unit dosage) of around 3 g for easy swallowing. For patients experiencing swallowing difficulties, the ketoester oral solution can be administered.

In accordance with the invention, insulin encompasses insulin extracted from natural animal tissues, recombinant human insulin, or insulin derivatives such as detemir insulin, glargine insulin, degludec insulin, OI338GT insulin, icodec insulin, etc. (while primarily used in injections, they may also be formulated into capsules).

Within the framework of the present invention, “ketoester” primarily denotes (R)-3-hydroxybutyrate-(R)-1,3-butanediol ester, its functional analogues or ketogenic substitutes, such as diester derivatives and bis(hexanoyl)-(R)-1,3-butanediol. Mixing ketoesters with other ketogenic substances can yield satisfactory ketogenic effects, such as KE1, which is a blend of ketoesters and sodium 3-hydroxybutyrate. The term “ketoester” within the present invention also encompasses mixtures of ketoesters and other ketogenic substances (such as pharmaceutically acceptable salts of 3-hydroxybutyric acid).

On the other hand, this invention also provides a method for controlling blood sugar in diabetes patients, which involves administering the kit of this invention to patients in need.

According to this invention, oral insulin enteric-coated capsules are taken at bedtime, while ketoester oral solution or soft capsules are taken before meals during the day (e.g., at least 0.5-1.0 hour prior, or exactly 1.0 hour, 2.0 hours, 3.0 hours, up to 4.0 hours prior).

The detailed procedural steps of the sugar management method described in this invention are as follows:
1. Take one insulin capsule at bedtime, with a dosage of 8 mg or less per capsule;
2. Consume a ketogenic diet for breakfast, or ingest approximately 12.5 g of ketoesters, followed by a normal diet half an hour later;
3. Maintain a balanced diet for lunch, ingesting 12.5 g of ketoesters 0.5-1 hour before meals;
4. For dinner, have a balanced diet and ingest 12.5 g of ketoesters 0.5-1 hour prior to the meal.

Consuming ketoesters aids in circumventing postprandial blood sugar spikes, and when combined with insulin capsules, this regimen compensates for the limitations in Oramed's clinical glycemic control framework, achieving enhanced glycemic management. The intake of ketoesters, which induce ketosis, varies significantly among individuals; thus, personal consumption levels must be adjusted based on blood ketone measurements to meet individual needs.

There are several benefits to using this combination regimen for blood sugar control in diabetes patients:
1. Compared to the effective oral insulin formulations developed by Tianhui Biotechnology, this regimen considerably diminishes insulin consumption and associated costs.
2. This regimen helps circumvent potential adverse effects associated with the activation of IGF receptors in intestinal cells when deploying large insulin quantities in Tianhui Biotechnology's formulations.
3. Compared to Tianhui Biotechnology's oral insulin, which solely provides insulin, ingestion of ketoesters can amplify insulin sensitivity and improve the condition of diabetes.
4. Approximately 60-70% of diabetes patients die from heart-related diseases. Ketoesters, being a superior energy source for both the brain and the heart, can enhance cardiac function when consumed by diabetes patients, thereby potentially reducing their mortality rate.
5. The combination of drugs in this kit can achieve enhanced effects (e.g., synergistic effects) compared to the administration of oral insulin capsules or consumption of ketoesters alone.

### BRIEF DESCRIPTION OF THE DRAWINGS

FIG. 1 shows the effects of different oral insulin dosages on fasting blood glucose and glycated hemoglobin in diabetic rat models.

FIG. 2 shows the effects of different ketoester dosages on fasting blood glucose and glycated hemoglobin in diabetic rat models.

FIG. 3 presents the blood glucose and glycated hemoglobin effects of the combination drug vs. canagliflozin in diabetic rat models.

FIG. 4 shows the effects of reduced-dose oral insulin in the combination drug on blood glucose and glycated hemoglobin.

### DETAILED DESCRIPTION OF THE DRAWINGS AND THE PRESENTLY PREFERRED EMBODIMENTS

Considering that the majority of diabetes patients are currently diagnosed with type II diabetes, which is mostly caused by obesity, patients undergoing insulin treatment typically have some degree of pancreatic damage leading to insufficient insulin secretion or significant insulin resistance at the cellular level. To evaluate the efficacy of the combination drug, the inventor selected an animal model of type II diabetes induced by obesity-related insulin resistance and associated pancreatic damage.

### 1. Establishment of the Animal Model

A study in Aging (Albany NY). 2021 Mar. 15; 13(5):7691-7706., indicates that diabetes is associated with aging. The outcomes of using animals of different ages to establish diabetes models vary significantly; therefore, selecting older animals for testing is more representative of actual conditions. The inventor opted for 12-month-old male SPD rats (weighing between 540 grams and 560 grams) for the experiment. These rats were fed a high-sugar, high-fat diet (10% lard, 2.5% cholesterol, 1.0% pig bile salt, 20% sucrose, and 66.5% conventional feed). After one month, insulin resistance was induced, followed by intravenous injection of STZ to inflict damage to the pancreatic beta-cells. The diet continued for an additional 12 weeks. Finally, rats with a fasting blood sugar level exceeding 10 mmol/L were selected, and their fasting blood sugar levels were measured for 3 consecutive times every 3 days. Rats maintaining stable fasting blood sugar levels above 10 mmol/L were chosen. Each group comprised 5 animals, and a standard diet (18.0% fat, 24.0% protein, and 58.0% carbohydrates) was administered during the drug intervention period. Given that the lifespan of glycated hemoglobin is approximately 3 months, the duration of drug intervention was established at 3 months to ensure precise measurement of post-intervention glycated hemoglobin. The diabetes animal model established as per this method is similar to the condition of most type 2 diabetes patients who are currently undergoing insulin therapy. A significance test was conducted on experimental animal data using one-way ANOVA, with a P-value of less than 0.05, where an asterisk (*) indicates a significant difference, and no asterisk indicates no significant differences.

### 2. Preparation of Oral Insulin Capsules and Ketoester Oral Solution

Referring to the method and ratios in Oramed's patent US10058593B2, oral insulin capsules were formulated by blending 8 mg of ordinary recombinant insulin, 150 mg EDTA, 150,000 units of aprotinin, and 150 mg of trypsin. These ingredients are mixed evenly with unsaturated fish oil, and then encapsulated in ultra-long type No. 9 enteric-coated capsules. Each capsule contained 5 U (units) of insulin, equivalent to 0.125 mg of insulin (40 U/mg insulin), resulting in the preparation of oral insulin enteric-coated capsules. The blank capsules for the control group maintained the identical composition and proportions, except for substituting the insulin with an equal amount of bovine serum albumin. The ketoester was diluted with flavored water to achieve a 50% concentration, while the control solution consisted of flavored water only. The method of gastric gavage was employed.

### 3. Determination of Oral Insulin Dosage

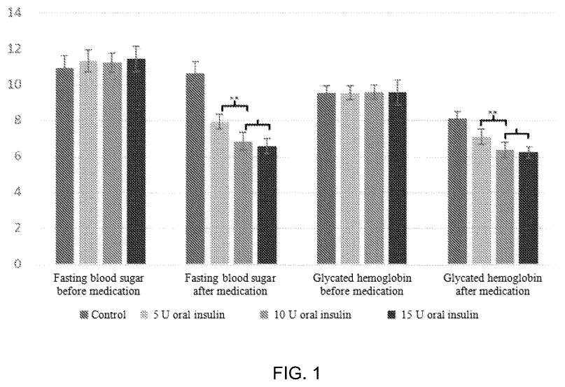

Referring to the literature in Biomaterial, issue 31, 2010, pp. 6849-6858, the oral insulin dosage for each animal was tested at levels of 5 U, 10 U, and 15 U. No food was provided after 5 PM, and oral insulin capsules were administered once daily at 10 PM. After three months of medication, blood was drawn from the tail to measure fasting blood sugar and glycated hemoglobin, with results expressed as the mean. The experimental results (refer to Table 1 and FIG. 1) demonstrated a significant difference in the effects between 5 U and both 10 U and 15 U, whereas there was little difference between 10 U and 15 U. The subsequent experiment utilized 10 U oral insulin.

TABLE 1
Effects of varying dosages of oral insulin on blood sugar and glycated hemoglobin levels

| | | Test group | Test group | Test group |
|---|---|---|---|---|
| | Control group | (5 U) | (10 U) | (15 U) |
| Fasting blood | 10.91 +/− 0.70 | 11.32 +/− 0.61 | 11.23 +/− 0.53 | 11.43 +/− 0.72 |
| sugar before | | | | |
| intervention | | | | |
| (mmol/L) | | | | |
| Glycated | 9.51 +/− 0.41% | 9.53 +/− 0.39% | 9.57 +/− 0.72% | 9.54 +/− 0.72% |
| hemoglobin | | | | |
| before | | | | |
| intervention | | | | |
| Number of blank | 3 | | | |
| capsules | | | | |
| Insulin- | | 1 | 2 | 3 |
| containing | | | | |
| capsules | | | | |
| After three | 10.63 +/− 0.63 | 7.93 +/− 0.42 | 6.84 +/− 0.51 | 6.58 +/− 0.41 |
| months of | | | | |
| intervention | | | | |
| Fasting blood | | | | |
| sugar | | | | |
| measurement | | | | |
| (mmol/L) | | | | |
| After three | 8.10 +/− 0.37% | 7.10 +/− 0.41% | 6.38 +/− 0.42% | 6.22 +/− 0.34% |
| months of | | | | |
| intervention | | | | |
| Glycated | | | | |
| hemoglobin | | | | |
| measurement | | | | |

### 4. 50% Ketoester Dosage Test

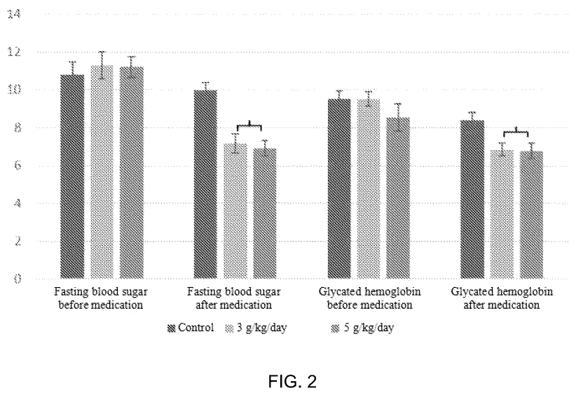

Feed 50% ketoester solution three times daily: at 8 am, 12 pm, and 5 pm. The total daily intake was selected as 3 g/kg/day or 5 g/kg/day, as referenced in BMC Anesthesiol. 2023; 23:p43. The results indicated (see Table 2 and FIG. 2) there was no significant difference between the two dosages. The subsequent experiment utilized 50% ketoester solution at 3 g/day/kg.

TABLE 2
Effects of consuming various amounts of ketoesters
on blood sugar and glycated hemoglobin levels

| | Control group | 3 g/kg/day | 5 g/kg/day |
|---|---|---|---|
| Fasting blood sugar | 10.78 +/− 0.67 | 11.28 +/− 0.71 | 11.19 +/− 0.54 |
| before administration | | | |
| (mmol/L) | | | |
| Glycated hemoglobin | 9.52 +/− 0.41% | 9.51 +/− 0.39% | 9.54 +/− 0.72% |
| before intervention | | | |
| Flavored water/total | 5 | | |
| daily amount (g) | | | |
| 50% ketoester/total | | 3 | 5 |
| daily amount (g) | | | |
| Fasting blood sugar | 9.97 +/− 0.42 | 7.15 +/− 0.51 | 6.90 +/− 0.41 |
| measurement after | | | |
| three months (mmol/L) | | | |
| Glycated hemoglobin | 8.37 +/− 0.41% | 6.82 +/− 0.34% | 6.75 +/− 0.42% |
| measurement after | | | |
| three months | | | |

### 5. Trial for Combination Drug of Oral Insulin Capsule and Ketoester

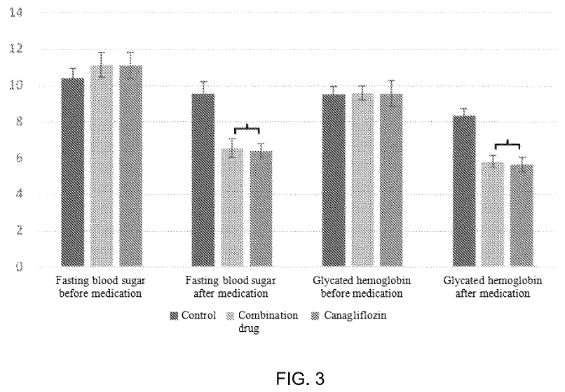

Combine the methods from sections 3 and 4 above to assess the efficacy of the combination drug of oral insulin and ketoester, while using canagliflozin, a drug currently used in clinical practice, as a control. The findings indicated (refer to Table 3 and FIG. 3) that there was no significant difference between the combination drug and canagliflozin.

TABLE 3
Effects of administering the combination drug and hypoglycemic drug
canagliflozin on fasting blood sugar and glycated hemoglobin levels

| | | Combination | |
|---|---|---|---|
| | Control group | drug group | |
| | (No drug | (Oral insulin/ | Canagliflozin |
| | intervention) | ketoester) | group |
| Fasting blood sugar | 10.41 +/− 0.53 | 11.12 +/− 0.68 | 11.08 +/− 0.72 |
| before intervention | | | |
| (mmol/L) | | | |
| Glycated hemoglobin | 9.53 +/− 0.41% | 9.57 +/− 0.39% | 9.56 +/− 0.63% |
| before intervention | | | |
| Empty capsules/flavored | 2 capsules/3 g | | |
| (g) | | | |
| Insulin-containing | | 2 capsules/3 g | |
| capsules/50% ketoester | | | |
| Dosage of hypoglycemic | | | Once a day in |
| drug (20 mg/kg) | | | the morning |
| Fasting blood sugar after | 9.56 +/− 0.64 | 6.56 +/− 0.51 | 6.38 +/− 0.41 |
| three months (mmol/L) | | | |
| Glycated hemoglobin | 8.31 +/− 0.42% | 5.81 +/− 0.34% | 5.63 +/− 0.42% |
| measurement after three | | | |
| months | | | |

*The measured values of fasting blood sugar and glycated hemoglobin in normal rats were 5.65+/−0.63 mmol/L and 3.83+/−0.49%, respectively.

### 6. The Synergistic Effect of Oral Insulin Capsules and Ketoester

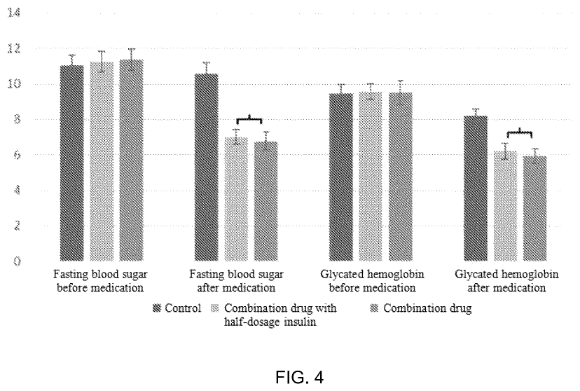

The bioavailability of oral insulin is generally 10-15%, and insulin accounts for the main cost in the combination drug. The inventor further explored whether the dosage of oral insulin in the combination drug could be further reduced when taken with ketoester. The experimental results (see table 4 and FIG. 4) indicated that reducing the dosage of oral insulin in the combination drug by half did not result in a significant difference in effect. The inventor is convinced that ketoesters enhance cell sensitivity to insulin, indicating a synergistic effect between oral insulin and ketoesters.

TABLE 4
Effects of the combination drug with reduced dosage of oral insulin
capsules on fasting blood sugar and glycated hemoglobin levels

| | | Combination drug | Combination drug |
|---|---|---|---|
| | Control group | (Oral insulin 5 U) | (Oral insulin 10 U) |
| Fasting blood sugar | 11.07 +/− 0.570 | 11.27 +/− 0.58 | 11.37 +/− 0.61 |
| before intervention | | | |
| (mmol/L) | | | |
| Glycated hemoglobin | 9.47 +/− 0.51% | 9.58 +/− 0.43% | 9.52 +/− 0.68% |
| before intervention | | | |
| Number of blank | 2 | | |
| capsules | | | |
| Insulin-containing | | 1 capsule/3 g | 2 capsules/3 g |
| capsules/50% ketoester | | | |
| solution | | | |
| After three months of | 10.57 +/− 0.63 | 7.03 +/− 0.42 | 6.79 +/− 0.51 |
| intervention | | | |
| Fasting blood sugar | | | |
| measurement (mmol/L) | | | |
| After three months of | 8.21 +/− 0.37% | 6.23 +/− 0.46% | 5.94 +/− 0.43% |
| intervention | | | |
| Glycated hemoglobin | | | |
| measurement | | | |

The above implementation steps provide a detailed description of the present invention. It should be noted that the above implementation steps are only for the purpose of illustrating the disclosed method of the present invention. Without departing from the spirit and essence of the present invention, those skilled in the art can design various alternative and improved solutions of the present invention, all of which should be understood as within the scope of protection of the present invention.

1. A drug combination kit for controlling blood sugar in diabetes patients, which comprises oral insulin capsules and ketoester oral solution or ketoester soft capsules.

2. The kit according to claim 1, wherein the dosage of oral insulin capsules is 2-20 mg, preferably 4-16 mg, more preferably 6-10 mg; the dosage of ketoester oral solution is 2-50 g, preferably 5-25 g, more preferably 10-15 g; or the dosage of ketoester soft capsules is 0.5-5 g, preferably 3-4 g.

3. The kit according to claim 2, wherein the insulin comprises natural insulin, recombinant insulin, or insulin derivatives (such as detemir insulin, glargine insulin, degludec insulin, OI338GT insulin, and icodec insulin).

4. The kit according to claim 2, wherein the ketoester comprises (R)-3-hydroxybutyrate-(R)-1,3-butanediol ester, its functional analogues or ketogenic substitutes (such as bis(hexanoyl)-(R)-1,3-butanediol), and mixtures of ketoesters and other ketogenic substances (such as mixtures of ketoesters and pharmaceutically acceptable salts of 3-hydroxybutyric acid).

5. A method for controlling blood sugar in diabetes patients, comprising administering the kit described in any one of claim 2 to patients in need.

6. The method according to claim 5, wherein oral insulin enteric-coated capsules are taken at bedtime, and ketoester oral solution or ketoester soft capsules are taken before meals during the day (e.g., at least 0.5-1.0 hour prior).
